(19) United States
(12) Patent Application Publication
(10) Pub. No.: US 2022/0313857 A1
(43) Pub. Date: Oct. 6, 2022

(54) AUTONOMOUS MOBILE DISINFECTION PLATFORM METHOD AND APPARATUS

(71) Applicant: Prescientx, Cambridge (CA)

(72) Inventor: Barry Hunt, Cambridge (CA)

(21) Appl. No.: 17/711,557

(22) Filed: Apr. 1, 2022

Related U.S. Application Data
(60) Provisional application No. 63/169,582, filed on Apr. 1, 2021.

(51) Int. Cl. A61L 2/24; A61L 2/10; A61L 2/22

(52) U.S. Cl. CPC A61L 2/24; A61L 2/10; A61L 2/22; A61L 2202/11; A61L 2202/14; A61L 2202/16

(57) ABSTRACT

An autonomous mobile disinfection platform for disinfecting an environmental surface in an environment, operable to autonomously navigate within the environment; locate the environmental surface using a set of optical image data; determine a surface type of the environmental surface; determine a desired ultraviolet dosage corresponding to the surface type, the desired ultraviolet dosage selected from a set of different predetermined dosages; direct the path adjacent the environmental surface; and direct the at least one ultraviolet radiation source to apply the desired ultraviolet dosage to the environmental surface.

### CROSS-REFERENCE TO RELATED PATENT APPLICATIONS

This application claims the benefit of U.S. Provisional Patent Application No. 63/169,582, filed on Apr. 1, 2021. The entire contents of U.S. Provisional Patent Application No. 63/169,582 is incorporated herein by reference for all purposes.

### FIELD

The specification relates generally to disinfection, particularly to an apparatus and method using an autonomous mobile disinfection platform.

### BACKGROUND

U.S. Pat. No. 8,999,238 to Kreitenberg purports to disclose a mobile body configured to travel over a surface inside an aircraft cabin. A source of UV radiation is mounted to the mobile body and configured to direct UV radiation to the surface at a predetermined dosage. At least two articulated arms are mounted to the mobile body, and UV lamps mounted respectively on the arms. The mobile body is a trolley or cart for negotiating an aircraft aisle.

U.S. Pat. No. 9,352,469 to Stewart purports to disclose a robot platform for remotely controlled and/or autonomous disinfection of technical facilities, including a drive mechanism configured to move the robot platform; a first disinfection module having a plurality of UV emitters disposed above the drive mechanism and selectively engagable shields which function to block the application of UV radiation in a specific direction, the disinfection module further having an articulating arm which has a second associated disinfecting module, the articulating arm has an actuator which can direct the emissions from the second disinfecting module, first disinfection module configured to disinfect the technical facility; and a position determination device configured to determine position data of the robot platform and a communication device configured to exchange control data and transmission of measurement and position data to an evaluation unit.

### SUMMARY

The following summary is intended to introduce the reader to various aspects of the applicant's teaching, but not to define any invention.

According to some aspects, there is provided an autonomous mobile disinfection platform for disinfecting an environmental surface in an environment, comprising a frame; a drive system coupled to the frame and operable to move the frame along a path at a speed; at least one sensor coupled to the frame and operable to acquire a set of position information indicative of a position of the mobile disinfection platform in the environment, the set of position information including a set of optical image data of the environment around the mobile disinfection platform acquired by an optical imaging system of the at least one sensor; at least one ultraviolet radiation source coupled to the frame and operable to direct an ultraviolet radiation output to a location adjacent the autonomous mobile disinfection platform; at least one processor communicatively coupled to the drive system to control the path and the speed, and communicatively coupled to the at least one sensor to receive the set of position information, the at least one processor operable to autonomously navigate the autonomous mobile disinfection platform within the environment, locate the environmental surface using the set of optical image data, determine a surface type of the environmental surface, the surface type being selected from a predetermined set of surface types, determine a desired ultraviolet dosage corresponding to the surface type, the desired ultraviolet dosage selected from a predetermined set of different dosages corresponding to the set of surface types, direct the path adjacent the environmental surface, and direct the at least one ultraviolet radiation source to apply the desired ultraviolet dosage to the environmental surface.

In some examples, the at least one processor is operable to maintain a relationship between the speed, a distance between the path and the environmental surface, and an ultraviolet output rate to ensure that the desired ultraviolet dosage is delivered to the environmental surface as the autonomous mobile disinfection platform passes the environmental surface.

In some examples, the at least one processor is operable to detect a human adjacent the mobile disinfection platform and prevent the at least one ultraviolet radiation source from directing the ultraviolet radiation output towards the human.

In some examples, the surface type is at least one of the group of a fabric surface and a smooth plastic surface.

In some examples, the autonomous mobile disinfection platform further comprises at least one storage device storing a set of map data of the environment, the at least one processor communicatively coupled to the at least one storage device to receive the set of map data and operable to use the set of map data in autonomously navigating the autonomous mobile disinfection platform around the environment, while using the set of position information to adapt to changes in the environment.

In some examples, the at least one ultraviolet radiation source includes at least two panels each including at least two ultraviolet lights, a first of the at least two panels directed in a first direction and a second of the at least two panels directed in a second direction different from the first direction.

In some examples, at least one of the at least two panels is moveable relative to the frame between a retracted position adjoining the frame and an extended position projecting an extended distance from the frame.

In some examples, the autonomous mobile disinfection platform further comprises at least one arm coupled to the frame and each carrying at least one of the at least one ultraviolet light source and operable to reposition the at least one of the at least one ultraviolet light source relative to the frame.

In some examples, the frame has a vertical frame axis and each of the at least one arm is operable to reposition the at least one of the at least one ultraviolet light source between a first radiation position directed transverse to the vertical frame axis and a second radiation position directed generally parallel to the vertical frame axis.

In some examples, the at least one arm is operable to active elevators and open doors.

In some examples, each of the at least one arm is an articulating arm.

In some examples, the at least one arm includes two arms operable to extend out opposite sides of the frame.

In some examples, the ultraviolet radiation output includes ultraviolet C light.

In some examples, the ultraviolet radiation output includes radiation with a wavelength of about 222 nm.

In some examples, the autonomous mobile disinfection platform further comprises a wagon module coupled to the frame to be towed by the frame.

In some examples, the autonomous mobile disinfection platform is a multimodal disinfection platform and includes a spray disinfector system operable to direct a spray disinfection output to the environmental surface adjacent the autonomous mobile disinfection platform.

In some examples, the autonomous mobile disinfection platform is a multimodal disinfection platform and includes an antimicrobial material application system operable to apply an antimicrobial material to the environmental surface adjacent the autonomous mobile disinfection platform.

In some examples, the autonomous mobile disinfection platform further comprises a microbial identification system operable to apply a chemical output to a surface which fluoresces in contact with microbes.

According to some aspects, there is provided a method of disinfecting an environmental surface of an environmental object in an environment, comprising receiving a set of optical image data from an autonomous mobile disinfection platform autonomously navigating within the environment, the set of optical image data including an image of the environmental surface in the environment; analyzing the set of optical image data to locate the environmental surface and determine a surface type of the environmental surface; determining a desired ultraviolet dosage corresponding to the surface type, the desired ultraviolet dosage selected from a set of different predetermined dosages; directing the autonomous mobile disinfection platform to travel adjacent the environmental surface; directing at least one ultraviolet radiation source mounted on the autonomous navigation platform to apply the desired ultraviolet dosage to the environmental surface from adjacent the environmental surface.

In some examples, the at least one ultraviolet radiation source is directed at the environmental surface to deliver the ultraviolet radiation output while the autonomous mobile disinfection platform is moving past the environmental surface at a speed, the method further comprising maintaining a relationship between the speed, a distance between the path and the environmental surface, and an ultraviolet output rate of the at least one ultraviolet radiation source to ensure that the desired ultraviolet dosage is delivered to the environmental surface.

### BRIEF DESCRIPTION OF THE DRAWINGS

The drawings included herewith are for illustrating various examples of articles, methods, and apparatuses of the present specification and are not intended to limit the scope of what is taught in any way. In the drawings:

FIG. 1 is a side view of a first example of an autonomous mobile disinfection platform;

FIG. 2 is a front view of the autonomous mobile disinfection platform of FIG. 1;

FIG. 3 is a rear view of a second example of an autonomous mobile disinfection platform with arms in a vertically extending position;

FIG. 4 is a rear view of the autonomous mobile disinfection platform of FIG. 3, with the arms in an intermediate position;

FIG. 5 is a rear view of the autonomous mobile disinfection platform of FIG. 3, with the arms in a raised horizontally extending position;

FIG. 6 is a rear view of the autonomous mobile disinfection platform of FIG. 3, with the arms in a lowered horizontally extending position;

FIG. 7 is a top perspective view of a third example of an autonomous mobile disinfection platform with arms in a rest position;

FIG. 8 is a bottom perspective view of the autonomous mobile disinfection platform of FIG. 7 with the arms in the rest position;

FIG. 9 is a top perspective view of the autonomous mobile disinfection platform of FIG. 7 with the arms in a lowered work position;

FIG. 10 is a top perspective view of the autonomous mobile disinfection platform of FIG. 7 with the arms in a raised work position;

FIG. 11 is a side view of a fourth example of an autonomous mobile disinfection platform; and

FIG. 12 is a flow chart of a method of disinfecting an environmental surface of an environmental object in an environment.

### DETAILED DESCRIPTION

Various apparatuses or processes will be described below to provide an example of an embodiment of each claimed invention. No embodiment described below limits any claimed invention and any claimed invention may cover processes or apparatuses that differ from those described below. The claimed inventions are not limited to apparatuses or processes having all of the features of any one apparatus or process described below or to features common to multiple or all of the apparatuses or process described below. It is possible that an apparatus or process described below is not an embodiment of any claimed invention. Any invention disclosed in an apparatus or process described below that is not claimed in this document may be the subject matter of another protective instrument, for example, a continuing patent application, and the applicants, inventors or owners do not intend to abandon, disclaim, or dedicate to the public any such invention by its disclosure in this document.

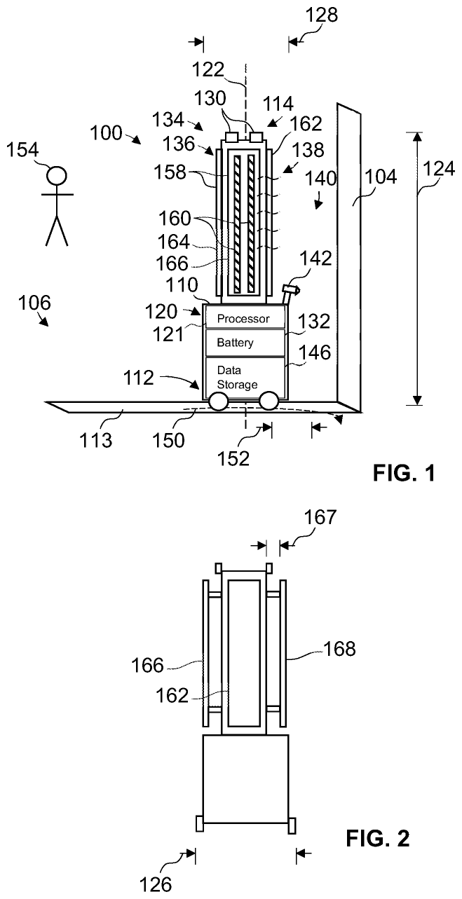

Referring to FIG. 1, illustrated is an example of an autonomous mobile disinfection platform 100 for disinfecting an environmental surface 104 in an environment 106.

In some examples, an autonomous mobile disinfection platform 100 is a self-driving vehicle that can plan its path to a destination location based at least on its current location and by referring to an electronic map of its environment. The autonomous mobile disinfection platform 100 can plan its path for conducting a mission or for collecting data from the environment. The autonomous mobile disinfection platform 100 can modify its planned path during operation. In some examples, the autonomous mobile disinfection platform 100 can modify its planned path based on data collected from its environment, such as in response to encountering an obstacle.

In the illustrated example, the autonomous mobile disinfection platform 100 includes a frame 110 supporting a drive system 112 and at least one sensor 114. The drive system 112 is coupled to the frame 110 and operable to move the frame 110 along a path at a speed. The illustrated drive system is a wheeled drive system for driving over a floor 113. In the illustrated example at least one processor 120 is communicatively coupled to the at least one sensor 114 and the drive system 112 to received sensed data and navigate the autonomous mobile disinfection platform 100 within the environment by controlling the path and the speed.

The illustrated example autonomous mobile disinfection platform 100 has a vertical axis 122. The illustrated example platform 100 has a height 124 greater than its width 126 (FIG. 2) and length 126. In the illustrated example, the height 124 of the autonomous mobile disinfection platform 100 is at least twice the width 126. A greater height may facilitate disinfecting raised surfaces such as the tops of furniture or upper portions of walls. A narrow width may facilitate fitting through doors and hallways. In the illustrated example, the width and length 126 are approximately equal. A narrow length may increase the mobility and navigability of the autonomous mobile disinfection platform 100.

The at least one sensor 114 is coupled to the frame 110 and, in some examples, is operable to acquire a set of position information indicative of a position of the autonomous mobile disinfection platform 100 in the environment 106. In some examples, the set of position information includes a set of optical image data of the environment around the mobile disinfection platform acquired by an optical imaging system 130 of the at least one sensor 114. Optical imaging data may be useful in identifying obstacles and/or in identifying surfaces to be disinfected.

One or more of the drive system 112, the at least one sensor 114, and a disinfection system 134 may require power. Power may be supplied from an on-board power supply. For example, power may be supplied by a battery 132 or other power storage device, or by an engine. Power may also be supplied from an external source, such as by way of a cord.

The autonomous mobile disinfection platform 100 includes at least one disinfection system 134 operable to apply a disinfection output to assist in disinfecting the environmental surface 104. In some examples, the disinfection system 134 is always on, such as turning on when the autonomous mobile disinfection platform 100 enters the environment 106 and staying on until the autonomous mobile disinfection platform 100 exits the environment 106. For example, the disinfection system may include an ultraviolet light that remains on continuously, or a misting or spraying system that continuously emits a mist or spray output. However, in some examples the disinfection system 134 is targeted, and is only applied to certain detected surfaces and/or changes its output for different detected surfaces.

In the illustrated example, the at least one disinfection system 134 is an ultraviolet disinfection system. The illustrated example autonomous mobile disinfection platform 100 includes at least one ultraviolet radiation source 136 coupled to the frame 110 and operable to direct an ultraviolet radiation output 138 to a location 140 adjacent the autonomous mobile disinfection platform 100. In some examples, the direct ultraviolet radiation output includes and/or is ultraviolet C light. Ultraviolet C light may be more germicidal than other subtypes of ultraviolet radiation.

In some examples, the at least one ultraviolet radiation source 136 is operable to kill microbes by delivering a predetermined output dosage to a surface on which the microbes are found, and thereby disinfect the surface. The predetermined output dosage may depend on the type of surface and the type of microbes.

In some examples, the autonomous mobile disinfection platform 100 is operable to deliver between 25 milijoules of ultraviolet C radiation per square centimeter and 1000 milijoules of ultraviolet C radiation per square centimeter to the environmental surface 104. In some examples, the autonomous mobile disinfection platform 100 is operable to deliver between 100 milijoules of ultraviolet C radiation per square centimeter and 1000 milijoules of ultraviolet C radiation per square centimeter to environmental surface 104. In some examples, the autonomous mobile disinfection platform 100 is operable to deliver at least 100 milijoules of ultraviolet C radiation per square centimeter to environmental surface 104. In some examples, the autonomous mobile disinfection platform 100 is operable to deliver at least 250 milijoules of ultraviolet C radiation per square centimeter to environmental surface 104.

In some examples, only a few milijoules per square cm is needed to kill pathogens. However, in some examples, more than a few milijoules per square cm is needed to kill pathogens. In some examples, a greater dose allows for more penetration of the radiation and/or accounts for obstructions of the radiation. In some examples, a dose above 1000 milijoules per square cm may damage some materials and/or be a wastefully high dose.

In some examples, the autonomous mobile disinfection platform 100 is a multimodal disinfection platform including more than one type of disinfection system 134. For example, the platform 100 may include at least two disinfection systems, and the at least two disinfection systems may include a spray disinfection system and an ultraviolet disinfection system. A spray disinfection system may be operable to direct a spray disinfection output to the environmental surface 104 adjacent the autonomous mobile disinfection platform 100. The spray disinfectant may be a germicidal liquid operable to kill germs it comes into contact with, and thereby disinfect surfaces on which it lands. A spray may be particularly useful in disinfecting non-planar surfaces and surfaces that are difficult to reach with ultraviolet radiation, such as nooks and crannies.

In another example of a multimodal system, the autonomous mobile disinfection platform 100 is a multimodal disinfection platform and includes an antimicrobial material application system operable to apply an antimicrobial material to the environmental surface 104 adjacent the autonomous mobile disinfection platform 100. The antimicrobial material may be a germicidal material that kills microbes after the microbes have been on the material for a predetermined length of time, and thereby disinfect a surface that incorporates or is overlaid with the antimicrobial material. An example of an antimicrobial material is the Aegis Microbe Shield™ made available by Microban International™.

The autonomous mobile disinfection system 134 may also include a microbial identification system 142 operable to apply a chemical output to a surface, the chemical output configured to fluoresce in contact with microbes. For example, the autonomous mobile disinfection system 134 may carry a spray system, such as a spray system used also to apply a spray disinfection output or a different spray system. The spray system may be used to spray a mist of liquid containing the chemical output onto a surface, and the autonomous mobile disinfection system 134 may be operable to image the surface to detect a level of fluorescence. An example of a microbial identification system is the OptiSolve Pathfinder™ system made available by Charlotte Products Ltd.™ of Peterborough, Ontario.

The autonomous mobile disinfection system 134 may also be operable to use the level of fluorescence to determine a level of contamination and a desired response, such as an extra cleaning step or a greater dose of a disinfection output.

The autonomous mobile disinfection platform 100 includes at least one processor 120. The illustrated at least one processor 120 is a single, onboard processor 121, but the at least one processor may optionally include more than one processor and/or a remote processor. For example, the at least one processor 120 may include an onboard processor communicatively linked with a remote processor such as a server or an administrative system.

The at least one processor 120 is communicatively coupled to the drive system 112 to control the path and the speed. The at least one processor 120 is also communicatively coupled to the at least one sensor 114 to receive the set of position information.

The at least one processor 120 is operable to autonomously navigate the autonomous mobile disinfection platform within the environment. For example, the at least one processor 120 may control the path and the speed of the drive system. The path may be preprogramed and/or adaptable, and the at least one processor may be coupled to a data storage device 146 storing a map of the environment and/or a preprogramed path, as discussed further below. The at least one data storage device 146 may be onboard and/or remote.

The at least one processor 120 is also operable to locate the environmental surface 104 using the set of optical image data. In some examples, an object recognition algorithm is applied to recognize an object in the set of optical image data. The object may have a surface to which ultraviolet radiation may be applied to disinfect the surface. For example, the object may be a wall or a door or a table, and the environmental surface may be a surface of a wall facing a rim, or a surface of a leg of a table, or a face of a door.

The at least one processor 120 is also operable to determine a surface type of the environmental surface, the surface type being selected from a predetermined set of surface types which may be stored on the data storage device. The at least one processor 120 is also operable to determine a desired ultraviolet dosage corresponding to the surface type, the desired ultraviolet dosage selected from a predetermined set of different dosages corresponding to the set of surface types, which may be stored on the data storage device. For example, there may be a predetermined dosage of the predetermined set of dosages for each surface type of the predetermined set of surface types.

A surface type may determine a desired dosage. In some examples, a greater dosage is applied to a high-priority surface or a high-touch surface that is more likely to be touched by individuals. A greater dose may assist in disinfecting a surface of a larger population of microbes. In some examples, a greater dosage is applied to a more textured surface. A greater dosage may result in increased radiation penetration. For example, a cloth surface may receive a greater dose than a smooth plastic or metal surface, and a bedrail may receive a greater dose than a baseboard.

In some examples, a surface type is determined using object recognition to identify the object that the surface is a part of. The at least one processor 120 may be operable to access records of object types and/or surface types stored on the at least one data storage device 146 and compare accessed data to the set of optical image data to determine an object type and/or surface type. In some examples, the set of different predetermined dosages is stored on the at least one data storage device 146, and the at least one processor 120 is communicatively coupled to the at least one data storage device 146 to access the set of different predetermined dosages and determine a desired ultraviolet dosage of the set corresponding to the surface type.

In some examples, the at least one data storage device stores a set of map data of the environment 106. The at least one processor 120 may be communicatively coupled to the at least one data storage device to receive the set of map data and operable to use the set of map data in autonomously navigating the autonomous mobile disinfection platform around the environment. For example, the at least one processor 120 may use the set of map data to generate the path 150 so that the path 150 brings the autonomous mobile disinfection platform 100 adjacent all the environmental surfaces 104 and/or all the environmental surfaces 104 that have been designated as needing to be cleaned. In some examples, the at least one processor 120 may use the map information while using the set of position information to adapt to changes in the environment 106. For examples, the set of position information may reveal an obstacle, and the autonomous mobile disinfection platform 100 may be operable to go around the obstacle and/or wait for the obstacle to move and/or adapt the plan to skip the environmental surface 104 that the autonomous mobile disinfection platform 100 is unable to reach due to the obstacle.

In some examples, the at least one processor 120 is operable to target particular surfaces. For example, the autonomous mobile disinfection platform 100 may be directed to target high-touch or high-priority surfaces such as door knobs, bedrails, and toilet seats. The at least one processor 120 may be operable to recognize objects that have been designated as high-priority or high-touch objects, and accordingly recognize one or more associated surface as a high-priority or high-touch surface. The autonomous mobile disinfection platform 100 may also or alternatively be operable to detect a high level of contamination on a surface and recognize the surface as a high-touch or high-priority surface.

The at least one processor 120 is operable to direct the path of the autonomous mobile disinfection platform 100 adjacent the environmental surface 104, and direct the at least one ultraviolet radiation source 136 to apply the desired ultraviolet dosage to the environmental surface 104.

In some examples, the at least one ultraviolet radiation source 136 is operable to deliver the ultraviolet output as the autonomous mobile disinfection platform 100 is in motion. Accordingly, the dosage delivered to the environmental surface depends on the distance 152 between the path 150 and the environmental surface 104 and the speed at which the autonomous mobile disinfection platform 100 passes the environmental surface 104. The dosage delivered may also depend on the ultraviolet output rate of the at least one ultraviolet radiation source 136. The at least one ultraviolet radiation source 136 may be operable to deliver the ultraviolet radiation output 138 at a variable or at a set ultraviolet output rate. In some examples, the ultraviolet output rate can be adjusted by the at least one processor 120. In some examples, the ultraviolet output rate is un-variable, and the dosage delivered to the environmental surface depends only on the distance 152 and the speed.

In some examples, the at least one processor 120 is operable to maintain a relationship between the speed of the autonomous mobile disinfection platform 100, the distance 152 between the path 150 and the environmental surface 104, and the ultraviolet output rate. The relationship may be selected to ensure that the desired ultraviolet dosage is delivered to the environmental surface 104 as the autonomous mobile disinfection platform 100 passes the environmental surface 104. The relationship may also be selected to minimize over-dosing the environmental surface 104. Over-dosing the environmental surface 104 may waste power. Particularly where the power supply is an on-board power supply, conserving power may increase the operating time of the autonomous mobile disinfection platform 100.

In some examples, the autonomous mobile disinfection platform 100 is adapted for operation around humans. In some examples, the autonomous mobile disinfection platform 100 operates on a schedule. For example, the at least one processor 120 may be operable to direct the autonomous mobile disinfection platform 100 in a scheduled cleaning through an environment. The scheduled cleaning may take place periodically according to a preset interval, such as a 24 hour interval or a 2 hour interval. The scheduled cleaning may also include periodic special cleaning, such as a regular cleaning every 24 hours and a deep cleaning every 30 days or every first calendar day or between patients in a hospital. A regular cleaning may include, for example, ultraviolet radiation and/or spray cleaning. A deep cleaning may include an additional type of disinfection or an additional length of cleaning. For example, a deep cleaning may include laying down an antimicrobial material and/or testing surfaces to determine a contamination level.

The autonomous mobile disinfection platform 100 may be operable to autonomously disinfect within the environment 106 without requiring all humans to leave first. The autonomous mobile disinfection platform 100 may be configured to work adjacent to a human.

In some examples, the at least one processor 120 is operable to detect a human 154 adjacent the autonomous mobile disinfection platform 100 and prevent the disinfection system from directing an output towards the human. For example, the at least one ultraviolet radiation source 136 may be prevented from directing the ultraviolet radiation output towards the human 154. For example, the at least one processor 120 may move a shield over one or more of the at least one ultraviolet radiation source 136 and/or turn off one or more of the at least one ultraviolet radiation source 136.

In some examples, the ultraviolet radiation output includes and/or is radiation with a wavelength of about 222 nm. Ultraviolet radiation with a wavelength of about 222 nm may be germicidal but less damaging to human tissue than other ultraviolet radiation. In some examples, the at least one ultraviolet radiation source 136 is a 222 nm radiation source and the ultraviolet radiation output 138 is 222 nm ultraviolet radiation. In some examples, the ultraviolet radiation output 138 is ultraviolet radiation with a wavelength between 215 nm and 230 nm. In some examples, the autonomous mobile disinfection platform 100 only carries 222 nm radiation sources. In some examples, the at least one processor 120 is operable to detect the human 154 and turn off or shield any of the ultraviolet radiation sources 136 that radiate ultraviolet radiation that is not about 222 nm. In some examples, the at least one processor 120 is operable to detect the human 154 and partially prevent ultraviolet radiation sources radiating ultraviolet radiation that is about 222 nm from directing the ultraviolet radiation output 138 towards the human 154. In some examples, the autonomous mobile disinfection platform 100 includes at least one lamp operable to radiate at about 222 nm and at least one lamp operable to radiate at about 254 nm.

In some examples, the at least one ultraviolet radiation source 136 includes one or more panels 158 each including at least two ultraviolet lights 160. In the illustrated example, the at least one ultraviolet radiation source 136 includes a plurality of panels 158. A first 162 of the panels 158 is directed forwards, a second 164 of the panels is directed rearwards, and a third 166 of the panels 158 is directed laterally. A fourth 168 (FIG. 2) of the panels 158 is also directed laterally, opposite the third 166 of the panels 158.

Referring to FIGS. 1 and 2, in some examples at least one of the panels 158 is moveable relative to the frame 110 between a retracted position adjoining the frame and an extended position projecting an extended distance 167 from the frame. In the illustrated example, the third 166 and the fourth 168 of the panels 158 are movable between the retracted position adjoining the frame (FIG. 1) and the extended position projecting an extended distance 152 from the frame (FIG. 2). In some examples, the at least one ultraviolet radiation source carried on an arm is operable independent of the position of the arm, for example it may be operable in an extended position and in a retracted position.

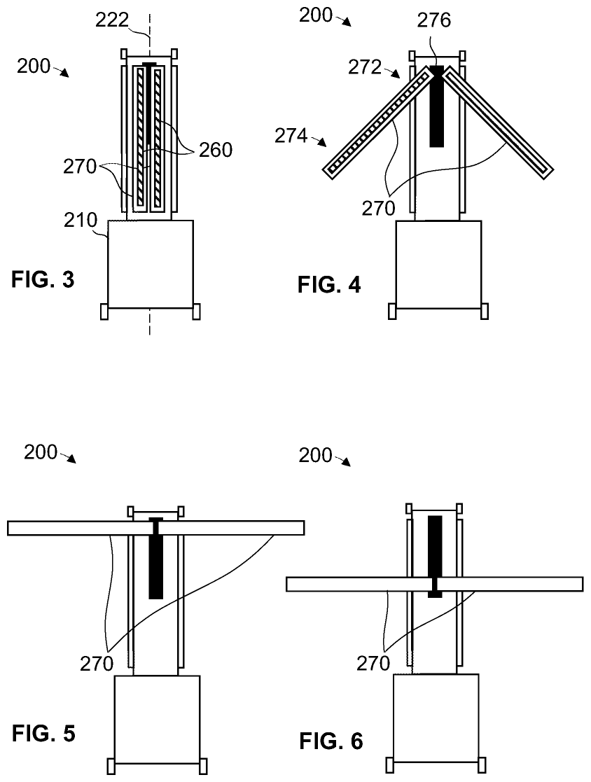

Referring now to FIGS. 3 to 6, illustrated is another example of an autonomous mobile disinfection platform 200. The autonomous mobile disinfection platform 200 is similar in many respects to the autonomous mobile disinfection platform 100, and like features are indicated with like reference numbers incremented by 100.

In some examples the autonomous mobile disinfection platform 200 includes one or more arms. Arms may carry ultraviolet light sources and/or other disinfection systems. Arms may allow the autonomous mobile disinfection platform 200 to reach surfaces that would otherwise be inaccessible or more difficult to reach. For example, a horizontal surface such as the top of a mattress or bed may be more difficult to disinfect from a position beside the bed than from a position above the bed. Arms may allow the autonomous mobile disinfection platform 200 to directed a disinfectant output such as ultraviolet light or disinfectant spray directly onto a surface, such as from a position proximate the surface and directing the disinfectant output substantially perpendicular to the surface.

In some examples, the autonomous mobile disinfection platform 200 includes at least one arm 270 coupled to the frame 210, the at least one arm 270 carrying at least one disinfection system. In the illustrated example, each arm 270 carries at least one ultraviolet light 260 and operable to reposition the corresponding ultraviolet light 260 relative to the frame 210.

In the illustrated example, the autonomous mobile disinfection platform 200 has a vertical axis 222 and each of the at least one arm 270 is operable to reposition the corresponding ultraviolet light 260 between a first radiation position (FIG. 3) directed transverse to the vertical axis 222 and a second radiation position (FIG. 5) directed generally parallel to the vertical axis 222.

In the illustrated example, each arm 270 is an elongated arm, and is secured to the frame 210 at a first end 272 at a pivotal mount (not shown). Each arm 270 can swing laterally out, with a second end 274 opposite the first end 272 swinging away from the vertical axis 222 of the autonomous mobile disinfection platform 200.

In the illustrated example, the first radiation position is a vertically extending position (FIG. 3), and the second radiation position is a horizontally extending position (FIG. 5). The vertically extending position may allow for easier storage and navigation of the autonomous mobile disinfection platform 200, as the arms 270 are held against the frame 210 along an entire length of the arms 270. The horizontally extending positon may allow for easier disinfection of a horizontal or inaccessible surface, such as the top of a bed or chair; allowing a disinfection output to be directed at the surface from an adjacent position and a desired angle. Directing a disinfection output from an adjacent position and a desired angle may increase the effectiveness of the disinfection output and/or reduce waste.

The arms 270 may also be maintained in one or more intermediate positons (an example shown in FIG. 4) between the vertically extending position and the horizontally extending position. An intermediate position may facilitate use with a greater range of surfaces.

The illustrated example arms 270 can also move vertically along a track 276 of the frame 210. The arms 270 can move between a raised position (FIG. 5) and a lowered position (FIG. 6). The vertical movement of the arms may facilitate use with a greater range of surfaces, such as with the horizontal surface on top of a bed and the horizontal surface on top of a chair, since the horizontal surface on top of the chair may be lower than the horizontal surface on top of a bed.

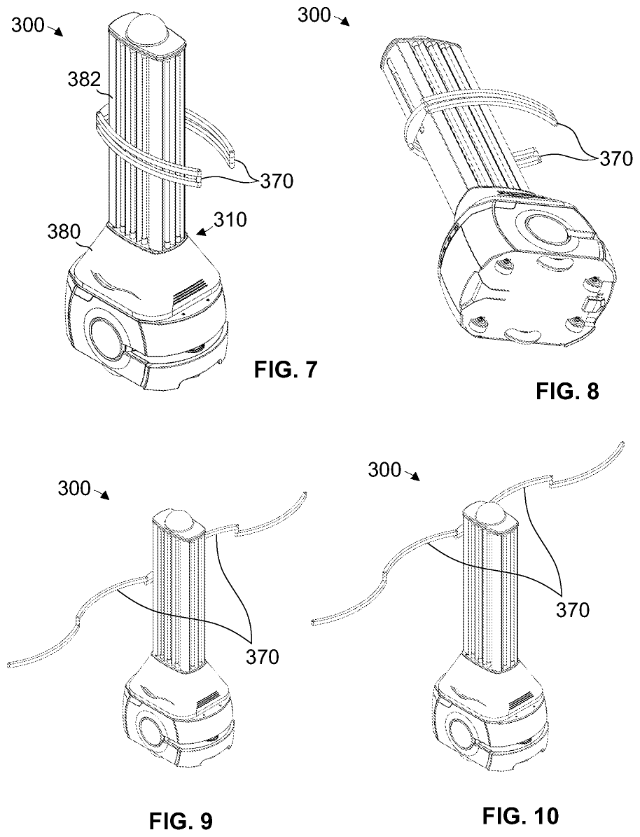

Referring now to FIGS. 7 to 10, illustrated is another example of an autonomous mobile disinfection platform 300. The autonomous mobile disinfection platform 300 is similar in many respects to the autonomous mobile disinfection platform 200, and like features are indicated with like reference numbers incremented by 100.

The illustrated autonomous mobile disinfection platform 300 includes a base 380 and a disinfection module 382. The base 380 is a self-driving platform, and the disinfection module 382 is mounted to the base 380 to be carried by the base 380. An example of a self-driving platform is the OTTO Materials Movement Platform™ from Clearpath Robotics Inc.™ of Kitchener, Ontario. The disinfection module 382 is a column shaped module and includes the at least one disinfection system 334, such as at least one ultraviolet source 336.

In some examples, an autonomous mobile disinfection platform includes at least one arm operable to active elevators and open doors. Where the autonomous mobile disinfection platform operates autonomously on a schedule, the autonomous mobile disinfection platform may need to be able to navigate through a building autonomously; moving between floors and rooms without human assistance. The illustrated example the autonomous mobile disinfection platform 300 includes articulating arms 370. The articulating arms 370 are operable to open doors and activate elevators. The articulating arms 370 may carry a disinfection system such as an ultraviolet light source or a spray disinfection system. Articulating arms 370 may also facilitate using the arms for activating elevators and opening doors.

In the illustrated example, the autonomous mobile disinfection platform 300 includes two arms 370 operable to extend out opposite sides of the frame 310. Each arm may move between a rest position (FIGS. 7 and 8) and a work position (FIG. 9) in which the arms 370 are extended out from the frame 310. In the illustrated example, the arms 370 are also vertically moveable along a track 376 of the frame 310 between a lowered position (FIG. 9) and a raised position (FIG. 10).

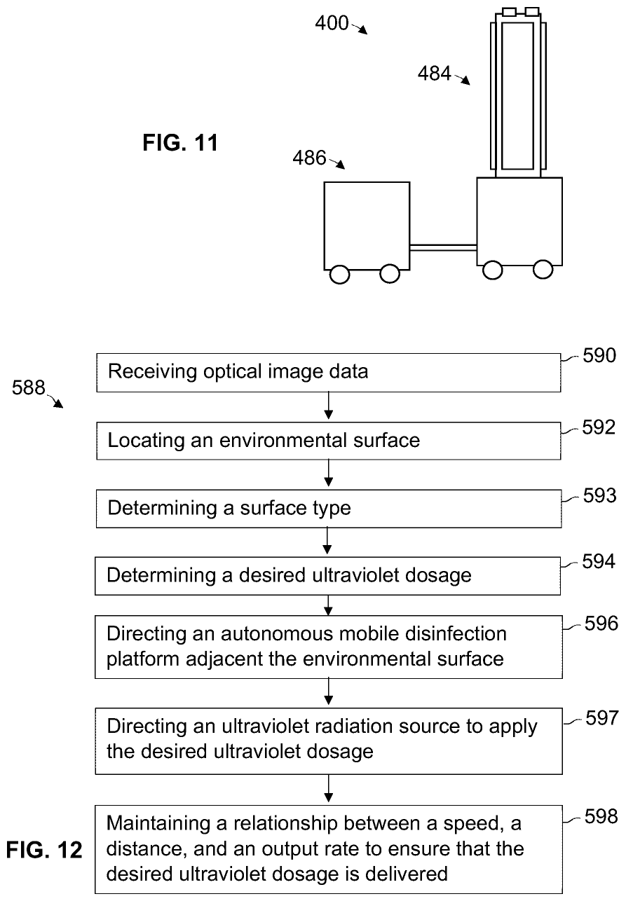

Referring now to FIG. 11, illustrated is another example of an autonomous mobile disinfection platform 400. The autonomous mobile disinfection platform 400 is similar in many respects to the autonomous mobile disinfection platform 200, and like features are indicated with like reference numbers incremented by 200.

An autonomous mobile disinfection platform may bring with it one or more supplementary platforms coupled to the autonomous mobile disinfection platform. Coupling a supplementary platform may permit the autonomous mobile disinfection platform to carry additional material with a single drive system while maintaining the dimensions of the autonomous mobile disinfection platform at a convenient size for fitting through doors and hallways. In some examples, the supplementary platform may carry a spray disinfection system while a main body carries an ultraviolet disinfection system.

The illustrated example autonomous mobile disinfection platform 400 includes a supplementary platform 486 towed by the frame 410. In the illustrated example, the mobile disinfection platform 400 includes a main body 484, including the frame 410. The supplementary platform 486 can be towed by the main body 484. The supplementary platform 486 may carry additional supplies, such as an extra power storage device or additional disinfection liquid for use in a disinfection spray system. The supplementary platform 486 may also carry one or more sensors and/or disinfection systems of its own.

The illustrated example autonomous mobile disinfection platform 400 tows a wagon module supplementary platform. The wagon module may be releasably coupled to the autonomous mobile disinfection platform 400 to allow the autonomous mobile disinfection platform 400 to operate without the wagon module.

Referring now to FIG. 12, illustrated is a method 588 of disinfecting an environmental surface of an environmental object in an environment. The method includes, at step 590, receiving a set of optical image data from an autonomous mobile disinfection platform autonomously navigating within the environment, the set of optical image data including an image of the environmental surface in the environment.

The method 588 includes, at step 592, analyzing the set of optical image data to locate the environmental surface and, at step 593, determine a surface type of the environmental surface, and, at step 594, determining a desired ultraviolet dosage corresponding to the surface type, the desired ultraviolet dosage selected from a set of different predetermined dosages.

The method 588 also includes, at step 596, directing the autonomous mobile disinfection platform to travel adjacent the environmental surface, and, at step 597, directing at least one ultraviolet radiation source mounted on the autonomous navigation platform to apply the desired ultraviolet dosage to the environmental surface from adjacent the environmental surface.

In some examples, the at least one ultraviolet radiation source is directed at the environmental surface to deliver the ultraviolet radiation output while the autonomous mobile disinfection platform is moving past the environmental surface at a speed. The method 588 may also include, at step 598, maintaining a relationship between the speed, a distance between the path and the environmental surface, and an ultraviolet output rate of the at least one ultraviolet radiation source to ensure that the desired ultraviolet dosage is delivered to the environmental surface.

1. An autonomous mobile disinfection platform for disinfecting an environmental surface in an environment, comprising:
a frame;
a drive system coupled to the frame and operable to move the frame along a path at a speed;
at least one sensor coupled to the frame and operable to acquire a set of position information indicative of a position of the mobile disinfection platform in the environment, the set of position information including a set of optical image data of the environment around the mobile disinfection platform acquired by an optical imaging system of the at least one sensor;
at least one ultraviolet radiation source coupled to the frame and operable to direct an ultraviolet radiation output to a location adjacent the autonomous mobile disinfection platform;
at least one processor communicatively coupled to the drive system to control the path and the speed, and communicatively coupled to the at least one sensor to receive the set of position information, the at least one processor operable to:
autonomously navigate the autonomous mobile disinfection platform within the environment,
locate the environmental surface using the set of optical image data,
determine a surface type of the environmental surface, the surface type being selected from a predetermined set of surface types,
determine a desired ultraviolet dosage corresponding to the surface type, the desired ultraviolet dosage selected from a predetermined set of different dosages corresponding to the set of surface types,
direct the path adjacent the environmental surface, and
direct the at least one ultraviolet radiation source to apply the desired ultraviolet dosage to the environmental surface.

2. The autonomous mobile disinfection platform of claim 1, wherein the at least one processor is operable to maintain a relationship between the speed, a distance between the path and the environmental surface, and an ultraviolet output rate to ensure that the desired ultraviolet dosage is delivered to the environmental surface as the autonomous mobile disinfection platform passes the environmental surface.

3. The autonomous mobile disinfection platform of claim 1, wherein the at least one processor is operable to detect a human adjacent the mobile disinfection platform and prevent the at least one ultraviolet radiation source from directing the ultraviolet radiation output towards the human.

4. The autonomous mobile disinfection platform of claim 1, wherein the surface type is at least one of the group of a fabric surface and a smooth plastic surface.

5. The autonomous mobile disinfection platform of claim 1, further comprising at least one storage device storing a set of map data of the environment, the at least one processor communicatively coupled to the at least one storage device to receive the set of map data and operable to use the set of map data in autonomously navigating the autonomous mobile disinfection platform around the environment, while using the set of position information to adapt to changes in the environment.

6. The autonomous mobile disinfection platform of claim 1, wherein the at least one ultraviolet radiation source includes at least two panels each including at least two ultraviolet lights, a first of the at least two panels directed in a first direction and a second of the at least two panels directed in a second direction different from the first direction.

7. The autonomous mobile disinfection platform of claim 6, wherein at least one of the at least two panels is moveable relative to the frame between a retracted position adjoining the frame and an extended position projecting an extended distance from the frame.

8. The autonomous mobile disinfection platform of claim 1, further comprising at least one arm coupled to the frame and each carrying at least one of the at least one ultraviolet light source and operable to reposition the at least one of the at least one ultraviolet light source relative to the frame.

9. The autonomous mobile disinfection platform of claim 8, wherein the frame has a vertical frame axis and each of the at least one arm is operable to reposition the at least one of the at least one ultraviolet light source between a first radiation position directed transverse to the vertical frame axis and a second radiation position directed generally parallel to the vertical frame axis.

10. The autonomous mobile disinfection platform of claim 8, wherein the at least one arm is operable to active elevators and open doors.

11. The autonomous mobile disinfection platform of claim 8, wherein each of the at least one arm is an articulating arm.

12. The autonomous mobile disinfection platform of claim 8, wherein the at least one arm includes two arms operable to extend out opposite sides of the frame.

13. The autonomous mobile disinfection platform of claim 1, wherein the ultraviolet radiation output includes ultraviolet C light.

14. The autonomous mobile disinfection platform of claim 13, wherein the ultraviolet radiation output includes radiation with a wavelength of about 222 nm.

15. The autonomous mobile disinfection platform of claim 1, further comprising a wagon module coupled to the frame to be towed by the frame.

16. The autonomous mobile disinfection platform of claim 1, wherein the autonomous mobile disinfection platform is a multimodal disinfection platform and includes a spray disinfector system operable to direct a spray disinfection output to the environmental surface adjacent the autonomous mobile disinfection platform.

17. The autonomous mobile disinfection platform of claim 1, wherein the autonomous mobile disinfection platform is a multimodal disinfection platform and includes an antimicrobial material application system operable to apply an antimicrobial material to the environmental surface adjacent the autonomous mobile disinfection platform.

18. The autonomous mobile disinfection platform of claim 1, further comprising a microbial identification system operable to apply a chemical output to a surface which fluoresces in contact with microbes.

19. A method of disinfecting an environmental surface of an environmental object in an environment, comprising:
receiving a set of optical image data from an autonomous mobile disinfection platform autonomously navigating within the environment, the set of optical image data including an image of the environmental surface in the environment;
analyzing the set of optical image data to locate the environmental surface and determine a surface type of the environmental surface;
determining a desired ultraviolet dosage corresponding to the surface type, the desired ultraviolet dosage selected from a set of different predetermined dosages;
directing the autonomous mobile disinfection platform to travel adjacent the environmental surface;
directing at least one ultraviolet radiation source mounted on the autonomous navigation platform to apply the desired ultraviolet dosage to the environmental surface from adjacent the environmental surface.

20. The method of claim 19, wherein the at least one ultraviolet radiation source is directed at the environmental surface to deliver the ultraviolet radiation output while the autonomous mobile disinfection platform is moving past the environmental surface at a speed, the method further comprising maintaining a relationship between the speed, a distance between the path and the environmental surface, and an ultraviolet output rate of the at least one ultraviolet radiation source to ensure that the desired ultraviolet dosage is delivered to the environmental surface.
